(19) United States
(12) Patent Application Publication
(10) Pub. No.: US 2024/0145058 A1
(43) Pub. Date: May 2, 2024

(54) BEHAVIOUR DETECTION USING WEARABLE DEVICES

(71) Applicant: League, Inc., Toronto (CA)

(72) Inventors: Ellsworth Marvin Campbell, III, Dunwoody, GA (US); Puneet Gupta, Toronto (CA); Kerry Weinberg, Waltham, MA (US)

(21) Appl. No.: 18/385,231

(22) Filed: Oct. 30, 2023

Related U.S. Application Data
(60) Provisional application No. 63/420,830, filed on Oct. 31, 2022.

(51) Int. Cl. G16H 20/00; G16H 40/67

(52) U.S. Cl. CPC G16H 20/00; G16H 40/67

(57) ABSTRACT

Biometric data or metrics of interest to a user are observed by wearable devices over a recurring time interval and aggregated into a representation of the user's baseline habits or patterns of behaviour. Present measurement of the same data or measures of interest within the recurring time interval provides a measure of the user's adherence to, or deviation from, the established habits or patterns as represented by a regularity score. Dynamic time warping barycenter averaging can account for time dependencies in the data or metrics of interest in both the baseline computation of past user habits and the characterization of the user's present behaviours. User regularity scores can be displayed to the user to both drive positive behavioural changes as well as initiate different health-related actions or recommendations for the user. Regularity scores can be computed repeatedly in line with long term changes in user habits and patterns of behavior.

### CROSS-REFERENCE TO RELATED APPLICATIONS

This application claims priority to U.S. Application Ser. No. 63/420,830, filed on Oct. 31, 2022, and entitled BEHAVIOUR DETECTION USING WEARABLE DEVICES, the entire contents of which are hereby incorporated by reference.

### TECHNICAL FIELD

The disclosure relates generally to techniques for processing physical or biometric user data collected from wearable devices and, more particularly, to detecting user habits and patterns of behaviours exhibited by the wearers of such devices.

### BACKGROUND

Wearable devices, such as fitness trackers and smart watches, have grown in popularity among consumers. Wearable devices may be used for different purposes including to monitor various user biometric signals, like heart rate or blood pressure, to track activities undertaken or completed by the wearer of the device, measure different metrics or parameters of physical user activity, such as steps taken, distance travelled, calories burned, heart rate, and time elapsed, to name a few examples, as well as many others. The data captured by a wearable device may generally be transmitted or uploaded to another connected device or program for further processing so as to provide the user with additional information about the user's health and lifestyle. Typically, but not necessarily, such information is intended to help the user to achieve or maintain a healthier lifestyle or to track progress made toward a health objective.

### SUMMARY

In general, the disclosure provides methods and systems for aggregating historical data related to a user variable or metric of interest into characterizations of the user's baseline patterns of behaviour or habits as well as the user's adherence to, or deviation from, from those baselines. The user variable or metric of interest may, for example, be observable by a wearable device. The characterization of baseline behaviour may generally be represented by one or more regularity scores that are computed from the aggregated historical data measured by the wearable device.

In at least one broad aspect, there is disclosed a method that includes receiving a data stream originated from the wearable device worn by the user, in which the data stream contains recurring measurements of an observed variable related to the user of the wearable device over an observation window. The data stream is segmented into a plurality of datasets, where each dataset contains time-series measurements of the observed variable corresponding to a different instance of a recurring time interval within the observation window. Two or more of the plurality of datasets are aggregated into a composite time-series representing a baseline measurement of the observed variable over past instances of the recurring time interval. A measure of similarity is computed between the composite time-series representation and another time-series representation containing measurements of the observed variable within a further instance of the recurring time interval. Based on the measure of similarity, a regularity score for the user is computed representing a normalized characterization of the observed variable in the further instance of the time interval in relation to the determined baseline measurement, and the computed regularity score may be transmitted to the user.

In at least one broad aspect, there is disclosed a method that includes receiving a data stream originated from a wearable device worn by a user, in which the data stream contains recurring measurements of an observed variable related to the user of the wearable device, and repeatedly processing the data stream as follows. The data stream may be repeatedly segmented into a dataset containing time-series measurements of the observed variable within an instance of a recurring time interval within an observation window. Two or more segmented datasets may be repeatedly aggregated into a composite time-series representing a baseline measurement of the observed variable over past instances of the recurring time interval. A measure of similarity may be repeatedly computed between the composite time-series representation and another time-series representation comprising measurements of the observed variable within a current instance of the recurring time interval. Based on the measure of similarity, a regularity score for the user may be repeatedly computed representing a normalized characterization of the observed variable in the further instance of the time interval in relation to the determined baseline measurement, and the computed regularity score for the current instance of the time interval may be repeatedly transmitted to the user.

In at least one broad aspect, there is disclosed a system that includes at least a wearable device and a host server. The wearable device generates a data stream containing recurring measurements of an observed variable related to a user of the wearable device. The host server is in communication with the wearable device and configured to: generate a composite time-series, representing a baseline measurement of the observed variable for a recurring time interval, by computationally aligning two or more time-series measurements of the observed variable corresponding to different instances of the recurring time interval within an observation window, and aggregating together the computationally aligned two or more time-series measurements of the observed variable; compute a measure of similarity between the composite time-series representation and another time-series representation comprising measurements of the observed variable within to a further instance of the recurring time interval; and transform the measure of similarity into a regularity score for the user representing a normalized characterization of the observed variable in the further instance of the time interval in relation to the determined baseline measurement.

Additional details of these and other aspects of the disclosed embodiments are provided below.

### BRIEF DESCRIPTION OF THE DRAWINGS

Reference will be made herein to the accompanying drawings, in which:

FIG. 1 shows an example data processing system suitable for implementing embodiments of the invention(s) disclosed herein.

FIG. 2 shows an example configuration of a client system that can be included in the data processing system of FIG. 1.

FIG. 3 shows an example configuration of an electronic device that can be included in the client system of FIG. 2.

FIG. 4 shows an example configuration of a host server that can be included in the data processing system of FIG. 1.

FIG. 5 shows an example method of processing data in accordance with embodiments of the invention(s) described herein.

For clarity and ease of description, like reference numerals will be used in the drawings to describe the same or like parts.

### DETAILED DESCRIPTION

The description which follows, and the embodiments described therein, are provided by way of illustration of example embodiments of the disclosed invention(s). These examples are provided for the purposes of explanation and disclosure and, unless stated explicitly or otherwise inferred from context, are not intended to limit or narrow any aspect or underlying principle of operation or functionality.

Wearable technologies, while capable of generating a rich amount of data related to a wide variety of user metrics or parameters of interest, do not necessarily output data in a form that is suitable or advantageous for human consumption. In some cases, a wearable device may generate raw or unprocessed data that, while still useful for different purposes in its own right, may not provide the user with complete context or deeper insight into the user's health or activities until that data has been subject to further processing and/or transformation. For example, a smart watch that implements a digital electrocardiogram (ECG) program might provide the wearer with a readout of their heart function at any given moment in time, without giving the user any valuable information or data related to trends, averages, or patterns in the user's historical heart function or overall heart health. Likewise the wearable device output data, without further processing, may provide no indication of the user's heart health relative to a mean or benchmark value ascertained from among a cohort population. Some wearable devices may also output data streams that are, for one reason or another, not inherently compatible with one another.

Embodiments of the invention(s) described herein provide a method and system for aggregating historical data related to a user variable or metric of interest, which may generally be observable by a wearable device or sensor, into characterizations of the user's baseline patterns of behaviour or habits, as well as the user's adherence to, or deviation from, that baseline pattern on an ongoing, e.g., day to day basis. As described herein, the user parameter or metric of interest may be tracked throughout a historical observation period of any chosen or suitable length that encompasses one or more recurring time intervals in which user behaviour or habits may be measured and characterized. For example, a time interval of interest may be a day of the week (e.g., to assess intra-week variation in sleep quality, steps or exercise behaviour, calorie burn or consumption, etc.), or any other relevant time interval, such as hours of the day, weeks of the month, months of the year, etc. A time interval of interest may also in some cases be characterized geographically or seasonally based on the location of the user and one or more environmental conditions experienced by the user over that time interval, such as temperature, levels of precipitation, hours of daily sunlight, and other factors.

Over the course of the observation period, the user metric or parameter of interest can be measured as a continuous or semi-continuous data stream originating from the wearable device and then segmented into separate datasets and recorded in a datastore, where each recorded dataset contains a time-series measurement of the parameter of interest over a different instance of a recurring time interval within the overall observation period. To generate a baseline measurement or determination of the observed variable for the given recurring time interval of interest, each recorded dataset corresponding to that interval of interest may be numerically or statistically processed and aggregated together into a measure of average value. In general, this process of data aggregation can be repeated or performed for any number of different recurring time intervals within the observation period, and for any number of different variables of interest that are observable by the wearable device(s). For example, a baseline measurement of some parameter or user metric of interest (e.g., sleep patterns, steps, calories) under observation can be computed for any particular day (or days) of the week, week (or weeks) of the month, hour (or hours) of the day, month (or months) of the year, season, etc., of interest that has been observed over the historical observation window.

Depending on the parameter or user metric of interest under observation, in some cases, it may be preferable or advantageous to employ historical observation widows of different lengths. In general, each different parameter or user metric may be observed over an observation window of a chosen length that is specific to that parameter or user metric. For example, sleep parameters may be observed over a 90 day observation window, while steps or calories burned are observed over a 30 days observation window. Use of differently sized observation windows may account for seasonality and other factors that can affect user behaviour and patterns.

To account for transient or dynamic characteristics or variations of the observed metric or parameter that are captured by the recorded time-series representations, the described embodiments may aggregate datasets together using or incorporating computational alignment techniques that take into account transient behaviours or time-dependencies of the observed variable, as opposed to other more static determinations, such as a as Euclidean distance or average. For example, in some cases, dynamic time warping (DTW) barycenter averaging may be utilized to generate an average or baseline measurement of the observed variable from the two or more input time-series measurements. Generating the composite representation of the observed variable using DTW barycenter averaging advantageously provides a mechanism to identify and preserve an average measure of recurring phenomena in the input time-series that have inherently comparable shapes or waveforms, but potentially different time dependencies or sequencing. An example of an observable metric that exhibits such time dependencies is steps taken, or calories burned, during periods of exercise such as gym workouts where the period of activity or exercise may have a generally similar duration and intensity, but variable start and finish times during the day. Detecting and characterizing patterns in the observable metric (user steps or calories) in this example means accounting for and interpreting time dependencies in the record signals (variation in start and end time).

By characterizing the user's baseline patterns of behaviour with respect to a given user metric or parameter of interest, the described embodiments may further allow for a characterization of the user's adherence to (or deviation from) that baseline. The characterization of baseline adherence can be made only once in reference to a single determination of the user's baseline or, alternatively, it can be made repeatedly on an ongoing basis, in which case the user's baseline measurement can also be determined repeatedly over time based on new or updated historical observations of the user variable. As new observations of the user metric or parameter are measured by the wearable device, the computation of baseline measurement can be re-performed and used for new characterizations of the user's baseline adherence.

According to the described embodiments, the user's adherence or deviation to their baseline can be numerically represented by a regularity score that is computed from the baseline representation (in some cases as updated over time) and present observations of the same user variable over the same recurring time interval of interest, such as day of the week. The regularity score can in some cases be defined so as to provide a normalized representation of a measure of similarity computed between the baseline measurement and the present observation, where the measure of similarity can be computed in some cases as a DTW distance. Advantageously, the regularity score can also be defined to have a non-zero lower bound and variable rate of change across its output range, in each case designed so that the regularity score generates feedback that selectively targets certain users over others that may comparatively benefit from positive feedback in relation to their behaviours and habit adherence. For example, a suitably designed sigmoid function may provide desirable characteristics for the computed regularity score, but other calculations of a regularity score may be possible as well.

Regularity scores may be transmitted and displayed to the user, for example, within a mobile application installed on the user's device(s) and/or used to drive other health-related actions or outcomes. For example, the regularity score can be used by a mobile device application to deliver curated content from within a specific health-campaign or program in which the user is enrolled, to deliver insights related to progress made through a health-related campaign, define milestones, trigger access to care or recommendations for the user concerning their health and the availability of different health care resources, and many other uses and applications generally without limitation.

As explained herein throughout, use of dynamic time warping in both the computation of a baseline measurement as well as the measure of similarly can advantageously preserve time-dependent features of the observed variable and thus produce a more complete description of user historical behaviours and patterns that does not inadvertently average out significant characteristics or features in the observed datasets. User habits or patterns within recurring time intervals of interest, such as days of the week, are therefore able to be characterized not by a single value, such as total steps taken, but by an average or representative time-series waveform taken across the entirety of the interval that can provide the user with deeper and more robust insights and information into their respective patterns and habits of behaviour.

Further details of various embodiments of invention(s), including at least one preferred embodiment, will now be provided with reference to the drawings.

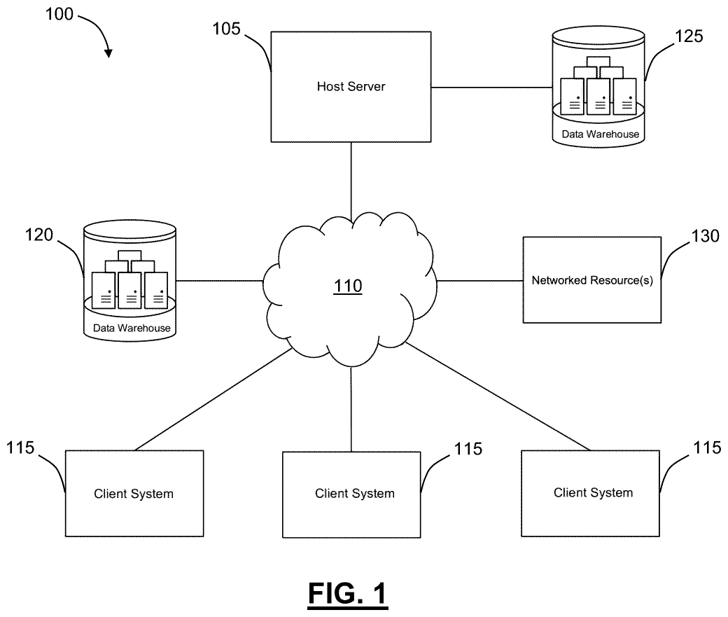

Reference is initially made to FIG. 1, which shows an example data processing system 100 that is suitable for implementing the embodiments disclosed herein. As shown, system 100 is based on a server-client architecture and may include a host server 105 that is connected to a network 110, which can be any local, wide area, or mobile network, including the Internet, cellular networks, and the like, or any combination of the foregoing. Two or more client systems 115 may be in communication with host server 105 through network 110 and used by individual users to access services, data, and other programs, mobile applications and functions that are implemented on host server 105. An example of three client systems is shown in FIG. 1, but in general there may be any number of client systems 115 corresponding to different users registered within data processing system 100 as described herein.

Client systems 115 may include one or more interconnected hardware devices that are configured for two-way network communication with host server 105 through an access program, such as a mobile or computer application, web browser, or other type or configuration of network interface. For example, client systems 115 may in some cases execute the front end of a user or client application including functions such as displays, visualizations, and presentations, user interfaces, data inputs and outputs, error alerts and notifications, alarms, and so on, as well as communicate with the application backend that is being implemented on host server 105. Likewise the application backend can perform data processing functions, such as data storage and retrieval, verification, authentication, processing business logic, security, and others.

In some cases, host server 105 may be or include any local or on-premises computer system configured with hardware and/or software resources that allow local or remote access by client systems 115. Alternatively, host server 105 may instead be implemented on a distributed or cloud-based environment, such as Google™ Cloud Services, Amazon™ Web Service, or Microsoft™ Azure™ Cloud Computing Platform, which may be located remotely at one or more third party data centres.

Host server 105 provides a computing and data processing platform that is operable to coordinate the operation of data processing system 100 and deliver programs, applications, media, packets, and other electronic content to client systems 115 across network 110. Host server 105 can be implemented using or including a client-server configuration in which the exchange of data between host server 105 and client systems 115 is controlled using remote procedure calls and API-level communication. In some cases, host server 105 may also incorporate encryption, such as SSL or other suitable security protocols, to secure communication and exchange of confidential and/or personal information with client devices 115.

One or more data warehouses or databases can be implemented within system 100 including, for example, both a networked (e.g., cloud-based) data warehouse 120 that is accessible to host server 105 over network 110 and/or a local data warehouse 125 that is dedicated to host server 105 directly. Each data warehouse 120,125 can be any suitable type of data storage system, such as a memory, disk, or monolithic or distributed database, which is configured to store information and data sets that are processed within a data processing layer of host server 105 as described herein.

In some cases, data warehouses 120,125 may be or include any local or on-premises computer system that is network-enabled or otherwise in communication with host server 105. Alternatively, either or both of data warehouses 120,125 may also be implemented on a distributed or cloud-based environment, such as Google™ Cloud Services, Amazon™ Web Service, or Microsoft™ Azure™ Cloud Computing Platform, which may be located remotely at one or more third party data centres.

The networked environment shown in FIG. 1 that provides an example data processing system 100 is for illustration purposes only. Different implementations and/or architectures will be possible to fit different use cases of a data processing system 100 that generates and delivers different content to client systems 115.

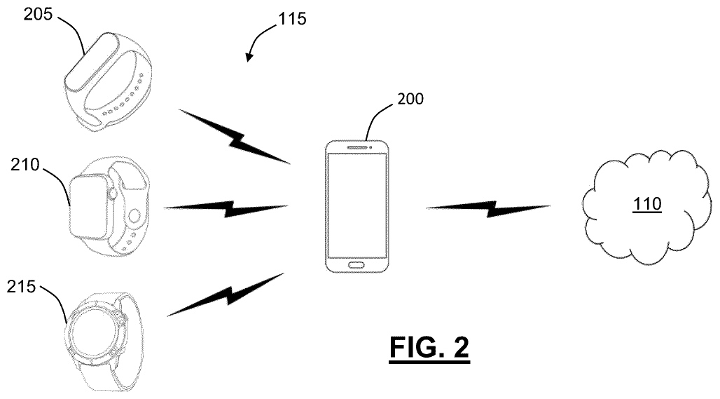

FIG. 2 shows an example configuration of a client system 115 including an electronic device 200 that is or can be coupled to a number of different peripheral devices with which device 200 is configured to interoperate. Electronic device 200 can, for example, be any mobile or handheld wireless communication device, such as a cellular phone, smart phone, wireless organizer, personal digital assistant (PDA), notebook computer, tablet device, or the like, which is capable of establishing connectivity to network 110 and on which the user may execute different programs or applications. Electronic device 200 can connect to network 110 either wirelessly, as in the above examples, but also in some cases using other non-wireless communication protocols, as for example in the case of a desktop computer, handheld electronic game device, digital camera, and so on, which may be connected by ethernet and other types of network connections.

In accordance with some embodiments, electronic device 200 can couple or be coupled to one or more different wearable devices such as a fitness tracker 205, a smart watch 210, or other type of function-specific wearable medical device 215, such as a heart rate or blood pressure sensor, which are designed to detect or measure a physical or biometric user parameter. When worn and activated by the user of device 200, wearable devices 205,210,215 can detect or monitor different metrics or user health parameters that are uploaded a connected program or application executing on mobile device 200 and which may then be transmitted over network 110 for processing by host server 105 as described further herein.

Example biometric parameters that wearable devices 205,210,215 can be configured to measure may relate to different physical or physiological data variables or metrics of the user, such as step count or another measure of distance travelled or energy expenditure, e.g., calorie burn, floors climbed and/or descended, heart rate, heartbeat waveform, heart rate variability, heart rate recovery, respiration, oxygen saturation, blood volume, blood glucose, skin moisture, location (e.g., via a GPS or GLONASS), and/or elevation. Other user biometric parameters that wearable devices 205,210,215 may be configured to measure may include user blood pressure, blood glucose levels, skin conductivity, skin and/or body temperature; muscle state (e.g., as measured by electromyography), brain activity (e.g., as measured by electroencephalography), weight, body fat percentages, caloric intake, measures of sleep function (including bed and rise times, sleep phases, sleep quality and/or duration), pH levels, hydration levels, and respiration rate, for example.

In some cases, wearable devices 205,210,215 can further be configured to measure or detect different environmental parameters, for example, barometric pressure, weather conditions (e.g., temperature, humidity, pollen count, air quality, rain/snow conditions, wind speed), light exposure (e.g., ambient light, UV light exposure, time and/or duration spent in darkness), noise exposure, radiation exposure, and/or magnetic field.

In addition to physical or biometric user parameters that are detected and measured by wearable devices 205,210,215, in some cases, electronic device 200 may also originate data or signals of interest that supplements or even substitutes for the data or signals generated by wearable devices 205,210,215. For example, wearable devices 205,210,215 may be monitoring user sleep activity when electronic device 200 registers that the user has received (answered) or placed a phone call, or some other interaction from the user. If wearable devices 205,210,215 had determined that the user is asleep during this period when that is not in fact the case, the data or signals from electronic device 200 may correct the erroneous signal reading from wearable devices 205,210,215.

Data or biometric parameters originating from wearable devices 205,210,215 can be communicated wirelessly to electronic device 200 using a short-range communication protocol such as through connection to a wireless access point, personal area network (PAN), Bluetooth pairing, or wireless local area network (WLAN). In some cases, wearable devices 205,210,215 can pair wirelessly with a mobile application or program executing on electronic device 200 that is configured to fetch or receive detected user biometric parameters from wearable devices 205,210,215.

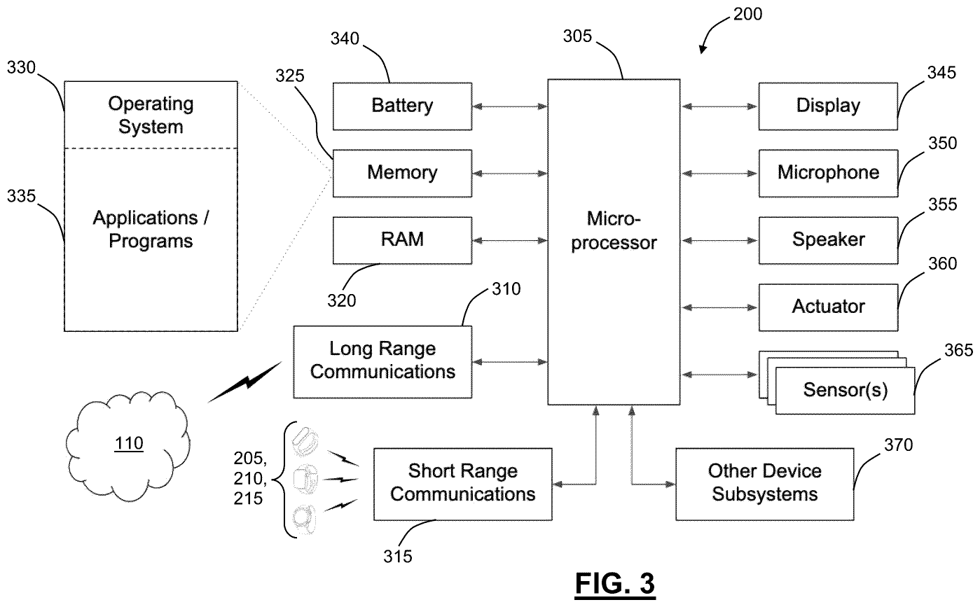

FIG. 3 illustrates an example configuration of electronic device 200 in accordance with the example embodiments disclosed herein. Electronic device 200 includes multiple integrated components, including a microprocessor 305 that controls the overall operation of electronic device 200. Long-range communication functions, including data and voice communications, are performed through a long-range communication subsystem 310, which receives messages or data packets from, and sends messages or data packs, to a wide area network, such as network 110 shown in FIG. 2. As described herein, network 110 can be any type of wide area network, including, but not limited to, a wired network, a data wireless network, voice wireless network, and dual-mode wireless networks that support both voice and data communications over the same physical base stations.

In accordance with the described embodiments, electronic device 200 may also include a short-range communication subsystem 315 that enables communication over local area or short-range wireless networks. For example, short-range communication subsystem 315 may include transmitters and receivers that are configured according to Bluetooth™, near field communication (NFC), and other communication protocols that are intended for communications between paired electronic devices over short distances. For example, electronic device 200 may use short-range communication subsystem 315 to enable interconnectivity with wearable devices such as fitness tracker 205 and smart watches 210 illustrated in FIG. 2.

Electronic device 200 may generally also include or be provided with random access memory (RAM) 320 and a memory 325, such as a hard drive, flash memory, or other persistent storage device, in which can be stored an operating system 330 and/or one or more functional applications or programs 335 that are executable by microprocessor 305. Applications or programs 335 can be either pre-loaded onto electronic device 200 or, in some cases, loaded or downloaded onto electronic device 200, for example, by the user accessing network 110 using long-range communications subsystem 310.

In some embodiments, electronic device 200 can be powered using an onboard battery 340, which can be a rechargeable battery designed for use in mobile electronic devices, such as a nickel-cadmium (NiCd), nickel metal hydride (NiMH), lithium-ion (Li-Ion), lithium polymer (Li-Po), and the like. Alternatively, in some embodiments, battery 340 can be replaced with and/or include a fixed power supply or adapter for a fixed power supply, such as an electrical grid.

When operated, a user can interact with electronic device 200 by way of one or more different input/output devices or components that can be included in different configurations of electronic device 200. For example, in some cases, electronic device 200 may also be equipped with a display 345, which may include a touch-sensitive screen, microphone 350, speaker 355, actuator 360, which can be an accelerometer, sensor(s) 365, and other device subsystems 370 generally without limitation.

Received signals, such as text messages, e-mail messages, instant messages, or web pages, may be processed by either long- and short-range communication subsystems 310,315 and then provided to microprocessor 305. Signals and data received at microprocessor 305 may then be routed to different subcomponents of device 200, such as an application or program 335, display 345, speaker 355, and so on in accordance with operation of the device 200. Users may also compose data items, such as text or email messages, using the touch sensitive display 345 or other input components, which messages can then be transmitted over network 110 using long-range communications subsystem 310.

In general, when operated by a user, electronic device 200 can be used to download and store any number and type of applications or programs 335 of the user's choosing. Often, but not necessarily, such applications or programs 335 can be retrieved and downloaded from an internet site or application repository, such as a mobile app store, to suit the user's interests. Some users may elect to download applications or programs 335 that are intended for the user to monitor and track general health and wellness.

In accordance with the described embodiments, a user may download an application or program 335 onto electronic device 200 that is compatible with one or more of wearable devices 205,210,215 shown in FIG. 2. Depending on the nature of the wearable device 205,210,215, the user may configure wearable device 205,210,215 to measure or track one or more different parameters or metrics of interest for the user. For example, the tracked parameter or metrics of interest may be any one or more of the examples that wearable device 205,210,215 is or may be configured to monitor.

For any user metric or parameter of interest that wearable device 205,210,215 may measure, a data stream containing those measurements may be transmitted by electronic device 200 through network 110 for processing by host server 105 in order to generate a baseline measurement of the user's habits or patterns and/or a regularity score indicative of the user's adherence to their established habits or pattern of behaviour. Host server 105 may then transmit the computed regularity score back to electronic device 200 for display to the user and/or for use by the user's mobile application to drive other health-related actions, recommendations, or outcomes.

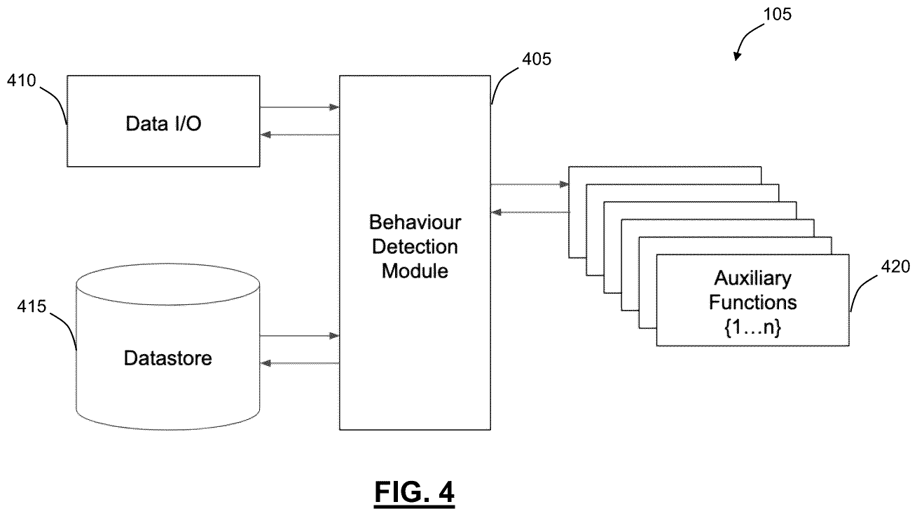

FIG. 4 illustrates an example configuration of host server 105 in accordance with the example embodiments disclosed herein. In the example shown, host server 105 includes a behaviour detection module 405 coupled to a data input/output module 410, datastore 415, and auxiliary functions 420. Data streams originating from a user mobile device (such as electronic device 200) or wearable device (such as wearable devices 205,210,215) are received at a data input/output module 410 and provided to behaviour detection module 405 where the data stream is segmented into different subsets each one containing time-series measurements of the observed user variable within a corresponding recurring time interval. For example, the recurring time interval can be defined as any quantity of minutes, hours, days, weeks, months, or some other measure of time, including based on geographical location or seasonality. In some cases, the recurring time interval may be defined as a calendar week or, alternatively, individual days of the week, such as every Monday or every Saturday, as examples.

As described herein, behaviour detection module 405 is configured to process the incoming data stream into one or more scores (referred to herein throughout as “regularity scores”) that represent a normalized characterization of the user's behaviour in the present with reference to a corresponding baseline measurement of the same user metric generated from observations taken in the past. The baseline measurement may be computed by behaviour detection module 405 to represent a “typical” or “average” measurement as ascertained from historical observations taken over an observation window of some chosen duration, which can vary for and be specific to different user metrics or variables of interest being observed. For example, an observation window of one length (e.g., 90 days) may be utilized for one metric or variable (e.g., sleep patterns), while a different length of observation window (e.g., 30 days) may be utilized for a different metric or variable (e.g., step count or calorie burn), as the case may be depending on the metric or variable in question. The chosen duration of the observation window may generally reflect assumptions made about long term variability of the observed metric or parameter including, in some cases, taking into account the duration of time in which seasonal variations around user behaviour may be expected to be exhibited or affected. Compared to raw data or measurements, the regularity score may be defined in such a way that the user is provided with a more intuitive indication of their adherence to existing behavioural patterns or habits.

In general, the user metrics or variables under observation may exhibit time-dependent variations to be accounted for and represented in the characterization of user habits or patterns of behaviour. Consider, for example, the steps behaviour of a hypothetical user that adheres to a regular or semi-regular weekly routine. Over the course of a typical week, this hypothetical user may adhere to a stable or relatively stable routine of office work Monday to Friday, gym visits three times during the work week (typically at lunch hour, but occasionally after work instead), and usually some exercise at some point on the weekend as well. While this may be the general pattern, week to week variation still may occur, as well as day to day variations during a given week. Gym workouts can take place on different days of the week or times of the day, workouts on the weekend can be missed, sometimes the user might take a day off work, and so on.

For this hypothetical user, characterizing typical or baseline behaviour for a given day using only static quantities (e.g., total step count for the day or calories burned) would not capture variations or patterns in behaviour that occur at different points in time throughout the day, such as periods of relatively intense activity (e.g., workouts) or inactivity (e.g., commutes, time at the office). At the same time, statistical averaging techniques that are based on Euclidean or root-mean-square distance may also produce an incomplete or distorted characterization of baseline behaviour by effectively averaging out time-dependent variations. These can present in different situations as distorted (duration may not be accurately represented) and/or phantom (features appearing in the baseline characterization did not actually occur in the input measurements) waveforms.

For example, in a given week, this hypothetical user may start their workout one day at noon, another day at 1 pm, and on a third day at 1:30 pm. Each workout may be 45 minutes long and, aside from the different start time, be comparable in terms of exertion and duration. The user may, for example, be attending a steps or spin class led by the same instructor on different days and times. Misleadingly, a Euclidean determination of average step count might suggest that a user's typical day involves a period of elevated activity lasting from noon and increasing in intensity until approximately 2:15 pm, whereas in fact a “typical” day for the user involves a consistent level of elevated step count experienced during workouts that lasts for more or less exactly 45 minutes, not two hours plus, but which has a variable start time falling somewhere between noon and 1:30 pm.

To account for and capture these and other time variations of interest in the observed variable, behaviour detection module 405 may compute the composite representation of the observed variable by employing statistical techniques that computationally align each input dataset automatically (or attempt to do so) according to waveform feature, while effectively filtering out null measurements, prior to aggregation. Computational alignment can involve both shifting (forward or backward) or scaling (compression or expansion) in time so that significant features or waveforms in the input time-series are aligned, or at least more aligned than without such processing. Some embodiments may advantageously employ DTW barycenter averaging for this purpose in order to better represent features of interest within the baseline representation of user habits or behaviour, although other statistical methods, such as Euclidean or root-mean-square averaging may also be employed in other embodiments.

Each regularity score computed by behaviour detection module 405 can be specific to a point or interval in time, such as the present, and for a particular user metric or parameter and time interval of interest that have been observed. In general, behaviour detection module 405 can compute a corresponding regularity score for any chosen number of user metrics or parameters, any chosen number of time intervals that recur within a historical observation window, and any chosen number of points in time, including repeatedly on a real-time or semi real-time basis as new data originating from wearable devices 205,210,215 is received at input/output module 410 and provided to behaviour detection module 405.

Data behaviour module 405 can compute a one-time regularity score for the user or, where provided by input/output module 410 with a continuous or semi-continuous data stream originating from the connected wearable device, a series of regularity scores for the observed variable in real-time or near real-time by advancing the historical observation window forward and repeating the computation of the regularity score(s) against updated baseline measurements and new observations of the user variable or metric. In this manner, a user may be provided with a continual, dynamic indication of how present behaviours compare to the user's established habits or patterns as ascertained from past observations of the same behaviour.

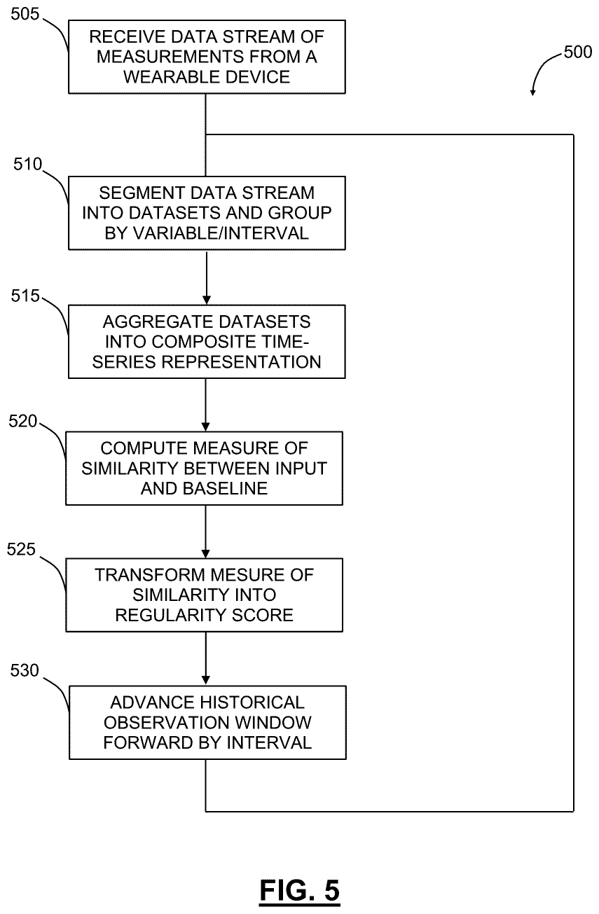

FIG. 5 illustrates an example method 500 of processing data in accordance with the described embodiments. Method 500 may be performed, for example, by behaviour detection module 405 shown in FIG. 4 to process input data streams into one or more regularity scores as described herein. The example implementation of method 500 shown in FIG. 5 is for illustrative purposes only. According to different implementations of method 500, individual steps or processes may be performed in a different order than as shown, by a different component, or omitted entirely, while additional steps and/or sequences not illustrated in the example method 500 may also be included as described herein generally without limitation.

For example, as illustrated, method 500 may begin at 505 by host server 105 receiving a continuous or semi-continuous data stream from an external source, which can be a wearable device 205,210,215 or a mobile device 200 to which a wearable device 205,210,215 is paired (as illustrated in FIG. 2). The continuous or semi-continuous data stream received by host server 105 may contain a time-series of input measurements of user metrics or parameters that are being observed by a wearable device 205,210,215 over a period of time that includes one or more recurring time intervals in which patterns or habits of user behaviour may be characterized. The input data stream may contain measurements of a single observed metric or, in some cases, multiple user metrics that are being observed simultaneously.

The data stream received at 505 may be segmented at 510 into different datasets (or sub-streams) according to both/either user variable or metric being observed and time interval of interest, as the case may be, such that datasets corresponding to the same variable/metric and the same interval of interest are associated together. Each subset (or sub-stream) segmented from the received data stream at 510 may contain an input time-series representation of the corresponding observed variable within the corresponding interval of interest, which, as noted herein, may generally recur over a period of time.

Datasets associated together at 510 may be aggregated at 515 into a composite time-series representing a baseline measurement of the corresponding observed variable as determined over all past instances of the recurring time interval within the observation window. As described herein, DTW barycenter averaging may be utilized in the generation of the composite time-series representation. The baseline measurement generated at 515 may, as a representation of user behavioural patterns or habits, be specific to the point in time at which it is computed and may generally be subject to long-term evolution as the user's behavior or habits may change.

At 520, a new input time-series segmented from the data stream is compared against the composite time-series representation generated at 515 and a measure of similarity is computed. The new input time-series segmented from the data stream may contain recent or current measurements of the same observed variable for the same time interval of interest as the composite time-series representation. As such, the comparison may implicitly be one of the user's adherence in the present time to habits or behavioural patterns that have been established in the past.

The comparison of the new input time-series measurements against the baseline representation at 420 can be performed using a chosen measure of similarity. For example, the measure of similarity may be a statistical averaging or distance measurement. In some cases, as described herein, the measure of similarity may be computed as a DTW distance between the input and composite time-series or some other equivalent or analogous measurement that incorporates time-series alignment (shifting and/or scaling) in the computation.

In some embodiments, the measure of similarity computed at 520 may be transformed at 525 into a regularity score that, by comparison, represents a normalized (and/or more intuitive) characterization of the user's present adherence to past habits or behavioural patterns. For example, the measure of similarity computed at 520 may, depending on the chosen distance measurement, generally in some embodiments be any real valued number greater than or equal to zero, where a zero-valued measure equates to a theoretical exact match between the input time series and the composite time-series representation generated at 515, and the larger the computed measure of similarity the greater the deviation between input and composite time series. Transforming the computed similarity measure into a regularity score at 525 can more intuitively characterize the user's present behaviour compared to baseline.

In some embodiments, a regularity score can be expressly defined to be bounded between chosen maximum and minimum values. For example, the regularity score computed at 525 can be bounded between 0 (corresponding to low or no similarity between the input time series and the baseline representation) and 100 (corresponding to an exact or near exact match). However, as an alternative, in some embodiments, a non-zero lower bound can be defined for the regularity score. For example, the regularity score can be bounded between, say, 40 (corresponding to low or no similarity) and 100 (corresponding again to an exact or near exact match). Defining a non-zero lower bound for the regularity score may be advantageous or beneficial in some cases to encourage or motivate user behavioural changes or adaptation, e.g., because a zero or effectively zero valued score may have the unintended consequence of demotivating or discouraging user behavioural change by conveying or giving the impression of too large a task to undertake, whereas a user may react more positively to a non-zero valued score that, while still low, provides the user with optimism and belief in improvement.

The measure of similarity may be transformed at 525 into a regularity score utilizing one or more different transformation functions with suitably defined input and output ranges. For example, a transformation function that produces an appropriately bounded output range over generally unbounded inputs, such as a sigmoid function, may be employed advantageously in some embodiments, but other functions may be utilized as well. Additional characteristics of its transfer function can make the use of a suitably defined sigmoid function advantageous. For example, sigmoids may tend to produce the largest rate of change toward the lower and midrange of the regularity score where users could benefit from positive feedback and reinforcement in the form of a rapidly increasing score. At the same time, a sigmoid will show a much lower rate of change toward the upper the upper end of the regularity score where users already show good adherence to habit and pattern and may therefore not benefit to the same degree from a changing score.

Following computation of the regularity score at 525, the observation window may be advanced forward at 530, at which point method 500 may branch back to 510 and repeat the computation of a regularity score based on new measurements of the observed variable taken from within the updated observation window. Over time, method 500 may repeatedly or continually produce a regularity score for the user that is assessed against an updated baseline representation of user behaviour that is tracking the long term evolution of user behavioural patterns and habits.

Referring back to FIG. 4, regularity scores computed by behaviour detection module 405 (e.g., using method 500 illustrated in FIG. 5) may be transmitted back to the user mobile device, e.g., electronic device 200 for display to the user and/or for other purposes. For example, behaviour detection module 405 may alternatively (or additionally) generate one or more health-related actions or recommendations for the user based on a computed regularity score. The type of actions or recommendations that can be delivered to the user may generally vary with the type(s) of application(s)/program(s) 335 (see FIG. 3) that the user has installed on electronic device 200. To generate recommendations and/or actions for the user, behaviour detection module 405 may communicate with datastore 415 and/or execute one or more auxiliary functions 420 as described herein below.

For example, in some embodiments, electronic device 200 may have installed application(s)/program(s) 335 that are related to the promotion of the user's general health and wellness. Application(s)/program(s) 335 may be configured, in some cases, to deliver various health-related activities or campaigns to the user based on user-inputted preferences, or health-related data of information about the user that has been collected by application(s)/program(s) 335, for example. Regularity scores computed by behaviour detection module 405 can be utilized by application(s)/program(s) 335 in different ways to drive the health-related content that is delivered to the user.

In some embodiments, regularity scores or other user habits data can be utilized by application(s)/program(s) 335 to define cohorts for health-related campaigns combined with segmentation logic that directs users to respective campaigns. For example, if a regularity score computed by behaviour detection module 405 indicates that a user has ineffective sleep habits, application(s)/program(s) 335 may, based on the value of the regularity score, recommend that the user complete a health-related campaign that targets improvements in sleep pattern.

In some embodiments, application(s)/program(s) 335 can recommend actions or health-related programs to the user of electronic device 200 based on a combination of two or more regularity scores generated by behaviour detection module 405. For example, a user's regularity scores may indicate that although a user spends an adequate or normal amount of time in bed (e.g., as ascertained from a sleep duration regularity score), the quality of the user's sleep is not effective (e.g., as ascertained from a time asleep regularity score). In such cases, application(s)/program(s) 335 may recommend a different health campaign to the user that targets activities for improving the effectiveness of sleep.

In some embodiments, the present value of a regularity score can be used by application(s)/program(s) 335 to recommend alterations to an ongoing health campaign in which the user is enrolled. For example, a health campaign may be configured with branching logic used to deliver different activities to the user by following different branches in the campaign. In some embodiments, the value of a regularity score computed by behaviour detection module 405 can be utilized within an application(s)/program(s) 335 to determine which branch of a campaign will be presented to the user. For example, within a campaign designed to target improvements in sleep quality, users with a large sleep quality regularity score may branch to one set of activities, while users with a low regularity score may branch to different activities.

In some embodiments, regularity scores computed by behaviour detection module 405 can be utilized within application(s)/program(s) 335 to provide insights to users on habit changes occurring while the user is enrolled in a campaign. For example, milestones related to habit or behavioural changes can be defined for a given campaign (e.g., maintain a certain level of regularity score, increase a regularity score by a certain amount). Milestones can be defined based on a single regularity score (e.g., sleep quality, sleep duration) or a regularity score combined with user-inputted information (e.g., sleep quality or duration with self-reported stress levels). Based on the input data, application(s)/program(s) 335 can be configured to make general recommendations and/or corrections for the user, or additionally, could provide the user with positive reinforcement, such as by recognizing or celebrating a milestone that is based on a regularity score.

In some embodiments, application(s)/program(s) 335 can combine user habits and behaviour data with progression through a health-related campaign in which the user is enrolled in order to generate recommendations for access to care. For example, based on a computed regularity score for a user, application(s)/program(s) 335 may recommend that a user make an appointment with a health care practitioner, seek advice on medications, or other actions that a user can take, as appropriate.

Although the disclosure has been presented with reference to certain specific embodiments, various modifications thereof will be apparent to those skilled in the art without departing from the scope of the disclosure. The scope of the claims should not be limited by the illustrative embodiments set forth in the examples, but should be given the broadest interpretation consistent with the description as a whole.

1. A method comprising:
receiving a data stream originated from a wearable device worn by a user, the data stream comprising recurring measurements of an observed variable related to the user of the wearable device over an observation window;
segmenting the data stream into a plurality of datasets, each dataset comprising time-series measurements of the observed variable corresponding to a different instance of a recurring time interval within the observation window;
aggregating two or more of the plurality of datasets into a composite time-series representing a baseline measurement of the observed variable over past instances of the recurring time interval;
computing a measure of similarity between the composite time-series representation and another time-series representation comprising measurements of the observed variable within a further instance of the recurring time interval;
based on the measure of similarity, computing a regularity score for the user representing a normalized characterization of the observed variable in the further instance of the time interval in relation to the determined baseline measurement; and
transmitting the computed regularity score to the user.

2. The method of claim 1, wherein the composite time-series representing the baseline measurement of the observed variable is generated using dynamic time warping (DTW) barycenter averaging of the plurality of datasets.

3. The method of claim 2, wherein the measure of similarity is computed as a DTW distance between the further time-series representation of the observed variable and the composite time-series representation.

4. The method of claim 1, wherein the regularity score is computed by transforming the measure of similarity using a sigmoid function.

5. The method of claim 4, wherein the regularity score has a non-zero lower bound.

6. The method of claim 1, further comprising recommending an action to the user based on the computed regularity score.

7. The method of claim 6, wherein the action is recommended as part of a health-related program in which the user is enrolled.

8. A method comprising:
receiving a data stream originated from a wearable device worn by a user, the data stream comprising recurring measurements of an observed variable related to the user of the wearable device; and
repeatedly,
segmenting the data stream into a dataset comprising time-series measurements of the observed variable within an instance of a recurring time interval within an observation window;
aggregating two or more segmented datasets into a composite time-series representing a baseline measurement of the observed variable over past instances of the recurring time interval;
computing a measure of similarity between the composite time-series representation and another time-series representation comprising measurements of the observed variable within a current instance of the recurring time interval;
based on the measure of similarity, computing a regularity score for the user representing a normalized characterization of the observed variable in the further instance of the time interval in relation to the determined baseline measurement; and
transmitting the computed regularity score for the current instance of the time interval to the user.

9. The method of claim 8, wherein the composite time-series representing the baseline measurement of the observed variable is generated using dynamic time warping (DTW) barycenter averaging of the two or more segmented datasets.

10. The method of claim 9, wherein the measure of similarity is computed as a DTW distance between the further time-series representation of the observed variable and the composite time-series representation.

11. The method of claim 8, wherein the regularity score is computed by transforming the measure of similarity using a sigmoid function.

12. The method of claim 11, wherein the regularity score has a non-zero lower bound.

13. The method of claim 8, further comprising recommending a series of actions to the user based on the computed regularity scores.

14. The method of claim 13, wherein the series of actions are recommended as part of a health-related program in which the user is enrolled

15. A system comprising:
a wearable device that generates a data stream comprising recurring measurements of an observed variable related to a user of the wearable device; and
a host server in communication with the wearable device, the host server configured to:
generate a composite time-series, representing a baseline measurement of the observed variable for a recurring time interval, by computationally aligning two or more time-series measurements of the observed variable corresponding to different instances of the recurring time interval within an observation window, and aggregating together the computationally aligned two or more time-series measurements of the observed variable;
compute a measure of similarity between the composite time-series representation and another time-series representation comprising measurements of the observed variable within to a further instance of the recurring time interval; and
transform the measure of similarity into a regularity score for the user representing a normalized characterization of the observed variable in the further instance of the time interval in relation to the determined baseline measurement.

16. The system of claim 15, wherein the host server is configured to:
generate the composite time-series representing the basement measurement of the observed variable using dynamic time warping (DTW) barycenter averaging of the two or more time-series measurements of the observed variable.

17. The system of claim 16, wherein the host server is configured to:
compute the measure of similarity as a DTW distance between the further time-series representation of the observed variable and the composite time-series representation.

18. The system of claim 15, further comprising:
a mobile device coupled to the wearable device and in communication with the host server, the mobile device configured to receive the regularity score transmitted from the host server and display the regularity score to the user within an application executing on the mobile device.

19. The system of claim 18, wherein the mobile device application is configured to recommend actions to the user based on computed values of the regularity score received from the host server.

20. The system of claim 19, wherein the actions are recommended as part of a health-related program executed by the mobile device application in which the user is enrolled.
